(19) United States
(12) Patent Application Publication
(10) Pub. No.: US 2026/0087621 A1
(43) Pub. Date: Mar. 26, 2026

(54) PROCEDURE SUPPORT APPARATUS AND PROCEDURE SUPPORT PROGRAM

(71) Applicant: FUJIFILM Corporation, Tokyo (JP)

(72) Inventors: Koji WAKI, Tokyo (JP); Yuko SATO, Tokyo (JP)

(73) Assignee: FUJIFILM Corporation, Tokyo (JP)

(21) Appl. No.: 19/330,754

(22) Filed: Sep. 16, 2025

(30) Foreign Application Priority Data
Sep. 25, 2024 (JP) 2024-166788

(51) Int. Cl. G06T 7/00; A61B 8/00; A61B 8/08

(52) U.S. Cl. CPC G06T 7/0012; A61B 8/085; A61B 8/4254; A61B 8/463; A61B 8/483; A61B 8/488; A61B 8/5261; G06T 2207/10132; G06T 2207/30096; G06T 2210/41

(57) ABSTRACT

A camera images a body surface portion in a vicinity of a tumor in a subject to form a captured image for display. A volume data acquisition unit acquires ultrasound volume data, corresponding to an ultrasound irradiation region including the tumor in the subject, which is formed based on reception signals obtained by transmitting and receiving ultrasound in the ultrasound irradiation region using an ultrasound probe in contact with the body surface portion of the subject. A reconstruction processing unit cuts out the ultrasound volume data in a cutout cross section perpendicular to a depth direction of the ultrasound volume data and including a procedure target region (region occupied by the tumor) to reconstruct the ultrasound volume data. A display controller displays the captured image for display and a reconstructed ultrasound image on a display.

This application claims priority to Japanese Patent Application No. 2024-166788 filed on Sep. 25, 2024 which is incorporated herein by reference in its entirety including the specification, claims, drawings, and abstract.

### BACKGROUND OF THE INVENTION

### 1. Field of the Invention

The present specification discloses improvements in a procedure support apparatus and a procedure support program.

### 2. Description of the Related Art

An ultrasound diagnostic apparatus that transmits ultrasound from an ultrasound probe in contact with a body surface of a subject toward the subject, receives reflected waves from the subject by the ultrasound probe, and forms an ultrasound tomographic image of the subject based on reception signals formed from the reflected waves is known.

In the related art, in a case in which a procedure (for example, surgery) is performed on a subject, an ultrasound diagnostic apparatus is used by an operator such as a doctor to check an inside of the subject.

For example, JP2024-52409A discloses an image processing apparatus that acquires a three-dimensional image (volume data) which is formed by an ultrasound diagnostic apparatus and which represents a procedure target tissue in a subject, and that renders the three-dimensional image from a side viewpoint of viewing a cut section for cutting the procedure target tissue from a side, which intersects a normal line of the cut section, in a visual line direction (that is, a side of the cut section) to form and display a side viewpoint image which is an ultrasound image in which the cut section is viewed from the side.

### SUMMARY OF THE INVENTION

In a case in which the procedure target tissue is present in the subject, the operator needs to incise the body surface of the subject before performing the procedure on the procedure target tissue. From the viewpoint of reducing invasiveness to the subject, it is desirable that an incision position on the body surface of the subject is in a vicinity of the procedure target tissue, such as directly above the procedure target tissue (in a depth direction).

It goes without saying that the operator cannot see the procedure target tissue before the body surface of the subject is incised. Therefore, in the related art, the operator performs work of checking the ultrasound tomographic image in which the procedure target tissue is shown before incision and specifying the incision position on the body surface of the subject based on the ultrasound tomographic image.

In the related art, it may take time and effort to specify the incision position from the ultrasound tomographic image, or it may be difficult to specify the incision position from the ultrasound tomographic image. For example, the ultrasound tomographic image, which is formed by bringing the ultrasound probe into contact with the vicinity of the incision position to transmit and receive the ultrasound to and from the procedure target tissue, represents the procedure target tissue viewed from the side in the visual line direction of the operator facing the incision position, and thus experience of the operator may be required in order to specify the incision position from the ultrasound tomographic image.

An object of a procedure support apparatus disclosed in the present specification is to allow an operator to easily identify an incision position on a body surface of a subject for performing a procedure on a procedure target tissue in the subject.

The present specification discloses a procedure support apparatus comprising: a captured image acquisition unit that acquires a captured image for display, which is obtained by imaging a body surface portion in a vicinity of a procedure target tissue in a subject; a volume data acquisition unit that acquires ultrasound volume data, corresponding to an ultrasound irradiation region including the procedure target tissue in the subject, which is formed based on reception signals obtained by transmitting and receiving ultrasound in the ultrasound irradiation region using an ultrasound probe in contact with the body surface portion; a procedure target region specifying unit that specifies a procedure target region that is occupied by the procedure target tissue in the ultrasound volume data; a reconstruction processing unit that forms a reconstructed ultrasound image by cutting out the ultrasound volume data in a cutout cross section perpendicular to a depth direction of the ultrasound volume data and including the procedure target region to reconstruct the ultrasound volume data; and a display controller that displays the captured image for display and the reconstructed ultrasound image on a display unit.

The procedure support apparatus may further comprise: a probe position and orientation information acquisition unit that acquires position and orientation information indicating a position and an orientation of the ultrasound probe in a case in which the ultrasound volume data is acquired, in which the captured image acquisition unit acquires a captured image for detection, which is obtained by imaging a probe detection marker attached to the ultrasound probe in contact with the body surface portion, the volume data acquisition unit acquires the ultrasound volume data including coordinate information indicating each position of the ultrasound volume data with reference to the position and orientation information in a camera coordinate system of a camera that has captured the captured image for display, and the display controller displays a partial capture image that is a portion of the captured image for display corresponding to the reconstructed ultrasound image on the display unit based on the coordinate information included in the ultrasound volume data.

The display controller may display the partial capture image and the reconstructed ultrasound image with matched display directions and display sizes thereof on the display unit.

The display controller may display a position indicator indicating a position in the partial capture image corresponding to a position of the procedure target region in the reconstructed ultrasound image on the display unit.

The partial capture image may include an image of a body surface characteristic object on a body surface of the subject, the procedure support apparatus may further comprise a distance measurement unit that measures a distance between the body surface characteristic object and the position indicated by the position indicator in a real space, based on a distance between the image of the body surface characteristic object and the position indicator in the partial capture image, and the display controller may display the distance measured by the distance measurement unit on the display unit.

The reconstruction processing unit may determine a position of the cutout cross section in the depth direction as a position at which an area of the procedure target region in the cutout cross section is largest.

The reconstruction processing unit may form the reconstructed ultrasound image based on partial volume data that is a part of the ultrasound volume data, including the cutout cross section and having a thickness in the depth direction of the ultrasound volume data.

The volume data acquisition unit may acquire the ultrasound volume data including a Doppler signal indicating a velocity of a blood flow of the subject at each position, the reconstruction processing unit may form a blood flow image representing the blood flow of the subject in the cutout cross section based on the Doppler signal, and form a reconstructed Doppler image in which the blood flow image is superimposed on the reconstructed ultrasound image, and the display controller may display the reconstructed Doppler image on the display unit.

Further, the present specification discloses a procedure support apparatus comprising: a volume data acquisition unit that acquires ultrasound volume data, corresponding to an ultrasound irradiation region including a procedure target tissue in a subject, which is formed based on reception signals obtained by transmitting and receiving ultrasound in the ultrasound irradiation region using an ultrasound probe in contact with a body surface portion in a vicinity of the procedure target tissue in the subject, the ultrasound volume data including a Doppler signal indicating a velocity of a blood flow of the subject at each position; a procedure target region specifying unit that specifies a procedure target region corresponding to the procedure target tissue in the ultrasound volume data; a reconstruction processing unit that forms a reconstructed ultrasound image by cutting out the ultrasound volume data including the Doppler signal in a cutout cross section perpendicular to a depth direction of the ultrasound volume data and including the procedure target region to reconstruct the ultrasound volume data, that forms a blood flow image representing the blood flow of the subject in the cutout cross section based on the Doppler signal, and that forms a reconstructed Doppler image in which the blood flow image is superimposed on the reconstructed ultrasound image; and a display controller that displays the reconstructed Doppler image on a display unit.

Further, the present specification discloses a procedure support program causing a computer to function as: a captured image acquisition unit that acquires a captured image for display, which is obtained by imaging a body surface portion in a vicinity of a procedure target tissue in a subject; a volume data acquisition unit that acquires ultrasound volume data, corresponding to an ultrasound irradiation region including the procedure target tissue in the subject, which is formed based on reception signals obtained by transmitting and receiving ultrasound in the ultrasound irradiation region using an ultrasound probe in contact with the body surface portion; a procedure target region specifying unit that specifies a procedure target region that is occupied by the procedure target tissue in the ultrasound volume data; a reconstruction processing unit that forms a reconstructed ultrasound image by cutting out the ultrasound volume data in a cutout cross section perpendicular to a depth direction of the ultrasound volume data and including the procedure target region to reconstruct the ultrasound volume data; and a display controller that displays the captured image for display and the reconstructed ultrasound image on a display unit.

With the procedure support apparatus disclosed in the present specification, the operator can easily identify the incision position on the body surface of the subject for performing the procedure on the procedure target tissue in the subject.

### BRIEF DESCRIPTION OF THE DRAWINGS

FIG. 1 is a diagram schematically showing a configuration of a procedure support system according to the present embodiment.

FIG. 2 is a diagram showing an example of a captured image for detection.

FIG. 3 is a diagram showing an example of a captured image for display.

FIG. 4 is a diagram schematically showing a configuration of an ultrasound diagnostic apparatus according to the present embodiment.

FIG. 5 is a conceptual diagram showing a concept of processing of forming ultrasound volume data.

FIG. 6 is a diagram showing a cutout cross section in the ultrasound volume data.

FIG. 7 is a diagram showing a plurality of cutout cross sections.

FIG. 8 is a diagram showing a data portion used to form a reconstructed ultrasound image.

FIG. 9 is a diagram showing an example of the reconstructed ultrasound image.

FIG. 10 is a diagram showing a display example of the captured image for display and the reconstructed ultrasound image.

FIG. 11 is a diagram showing a partial capture image in the captured image for display.

FIG. 12 is a diagram showing a display example of a position indicator.

FIG. 13 is a diagram showing a display example of a distance between a nipple and a recommended incision position.

FIG. 14 is a diagram showing an example of a reconstructed Doppler image.

FIG. 15 is a diagram showing a display example of the reconstructed Doppler image.

FIG. 16 is a diagram showing a display example of the captured image and the reconstructed Doppler image.

### DESCRIPTION OF THE PREFERRED EMBODIMENTS

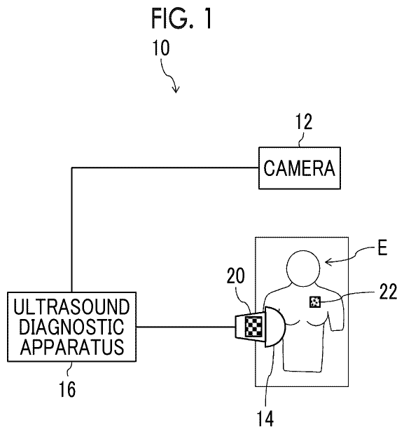

FIG. 1 is a diagram schematically showing a configuration of a procedure support system 10 according to the present embodiment. The procedure support system 10 includes a camera 12 and an ultrasound diagnostic apparatus 16, as a procedure support apparatus, including an ultrasound probe 14. The camera 12 and the ultrasound diagnostic apparatus 16 are connected to each other so as to be communicable with each other.

In the present embodiment, a probe detection marker 20 is attached to the ultrasound probe 14. The probe detection marker 20 is a marker for detecting a position and an orientation of the ultrasound probe 14. Further, a body surface detection marker 22 is attached to a body surface of a subject E. The body surface detection marker 22 has a different pattern from the probe detection marker 20, and is a marker for detecting a position and an orientation of the body surface of the subject E. Examples of the probe detection marker 20 and the body surface detection marker 22 include an augmented reality (AR) marker.

The camera 12 includes, in addition to a lens and an image sensor, a processor including a central processing unit (CPU) and the like, a communication interface including a network adapter and the like, and the like. The camera 12 images the ultrasound probe 14 (specifically, the probe detection marker 20) and the subject E (specifically, the body surface detection marker 22). The position and the orientation of the ultrasound probe 14 are detected by the ultrasound diagnostic apparatus 16 as described later by using a captured image obtained by such imaging. In the present specification, such a captured image will be referred to as a captured image for detection.

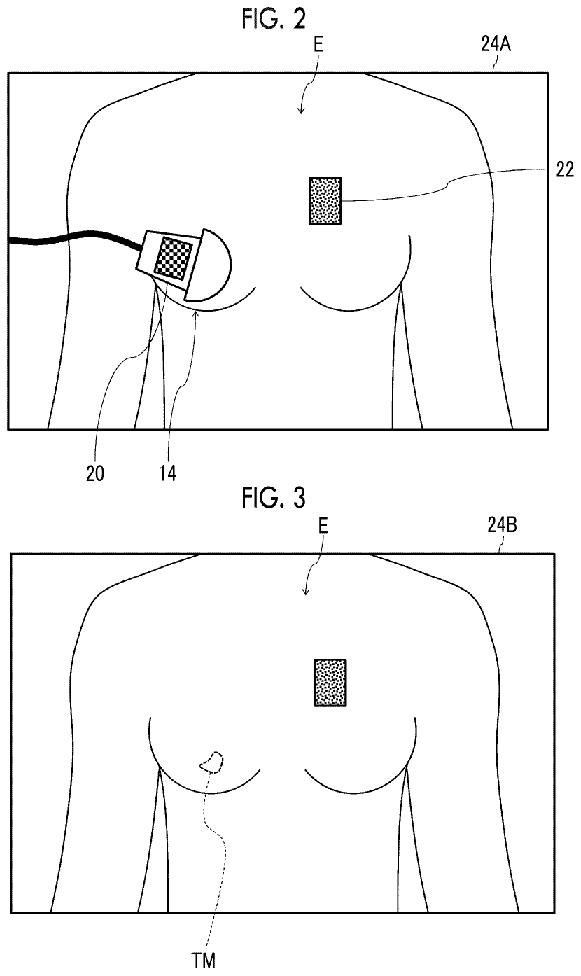

FIG. 2 is a diagram showing an example of a captured image for detection 24A. As described above, the image of the probe detection marker 20 and the image of the body surface detection marker 22 are included in the captured image for detection 24A. The ultrasound diagnostic apparatus 16 can detect the position and the orientation of the ultrasound probe 14 by analyzing the image of the probe detection marker 20 shown in the captured image for detection 24A. Further, the ultrasound diagnostic apparatus 16 can detect the position and the orientation of the body surface of the subject E by analyzing the image of the body surface detection marker 22 shown in the captured image for detection 24A. Details of processing of detecting the positions and the orientations of the ultrasound probe 14 and the subject E will be described later.

Further, the camera 12 images a body surface portion in a vicinity of a procedure target tissue (details will be described later) in the subject E. In the present specification, such a captured image will be referred to as a captured image for display.

FIG. 3 is a diagram showing an example of a captured image for display 24B. Although the present disclosure is not limited to this, in the present embodiment, an example will be described in which the procedure target tissue is a tumor TM in a breast of the subject E. Therefore, in the present embodiment, as shown in FIG. 3, the captured image for display 24B includes an image of a body surface portion in the vicinity of the tumor TM, that is, a periphery of the breast. In particular, the captured image for display 24B is an image in which the periphery of the breast is imaged from a front side of the subject E. In a case in which the body surface portion in the vicinity of the procedure target tissue is shown in the captured image for detection 24A, the captured image for detection 24A may be used as the captured image for display 24B.

In the present specification, the concepts including the captured image for detection 24A and the captured image for display 24B will be simply referred to as a captured image. A position and an orientation of the camera 12 in a case in which the captured image for detection 24A is captured and a position and an orientation of the camera 12 in a case in which the captured image for display 24B is captured are set to be the same as each other.

The captured image is formed by the image sensor of the camera 12, and the captured image is transmitted to the ultrasound diagnostic apparatus 16 via the communication interface of the camera 12.

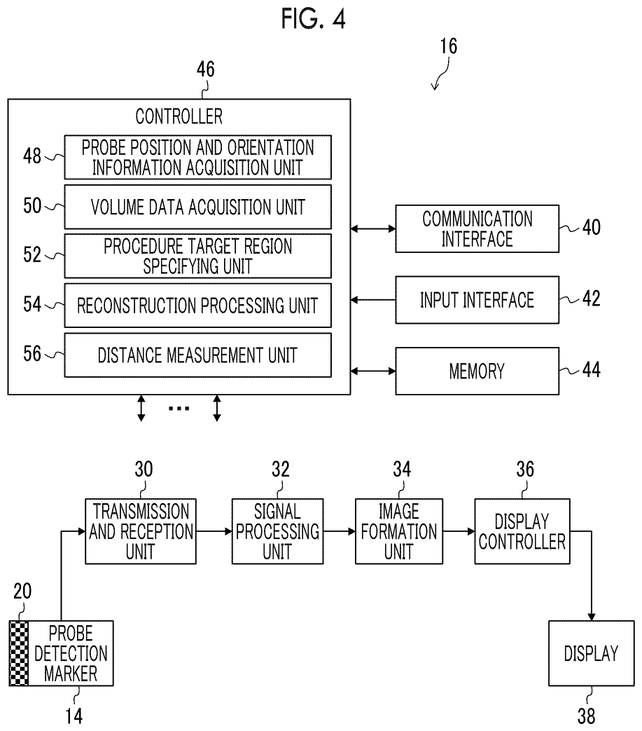

FIG. 4 is a diagram schematically showing a configuration of the ultrasound diagnostic apparatus 16. The ultrasound diagnostic apparatus 16 is a medical apparatus installed in a medical institution, such as a hospital.

The ultrasound probe 14 is a device that transmits and receives ultrasound to and from the subject E. The ultrasound probe 14 includes a transducer element array consisting of a plurality of transducer elements that transmit and receive the ultrasound to and from the subject E. The transducer element array is composed of the plurality of transducer elements arranged in one direction (array direction). In a case in which a transmission signal is supplied to each transducer element from a transmission and reception unit 30, which will be described later, each transducer element generates the ultrasound. Specifically, the ultrasound probe 14 scans an ultrasound beam on a plane (scanning plane) parallel to the array direction.

As described above, the probe detection marker 20 is attached to the ultrasound probe 14.

The transmission and reception unit 30 transmits the transmission signal to the ultrasound probe 14 (specifically, each transducer element of the transducer element array) under control of a controller 46 described later. As a result, the ultrasound beam is scanned on the scanning plane from the ultrasound probe 14. In addition, the transmission and reception unit 30 receives a reception signal from each transducer element that has received reflected waves from the subject E. The transmission and reception unit 30 includes an adder and a plurality of delay devices corresponding to the respective transducer elements and performs phase alignment and addition processing of aligning and adding phases of the reception signals from the transducer elements using the adder and the plurality of delay devices. As a result, a reception beam signal is formed in which pieces of information indicating a signal intensity of the reflected wave from the subject E are arranged in the depth direction of the subject E. A plurality of reception beam signals corresponding to one scanning plane constitute frame data.

A signal processing unit 32 performs various types of signal processing including filter processing of applying a bandpass filter, detection processing, and the like, on the reception beam signal from the transmission and reception unit 30.

An image formation unit 34 forms an ultrasound tomographic image (B-mode image) representing a cross section (particularly, the scanning plane of the ultrasound beam) of the subject E based on the reception beam signal on which the signal processing has been performed in the signal processing unit 32.

A display controller 36 performs control of displaying the ultrasound tomographic image formed by the image formation unit 34 on a display 38. In addition, as will be described in detail later, the display controller 36 displays a reconstructed ultrasound image and the captured image for display 24B formed by the controller 46 on the display 38.

The display 38 as a display unit is a display device configured by, for example, a liquid-crystal display or an organic electroluminescence (EL).

The transmission and reception unit 30, the signal processing unit 32, the image formation unit 34, and the display controller 36 included in the ultrasound diagnostic apparatus 16 are configured by a processor. The processor includes at least one of a general-purpose processing device (for example, a CPU or the like) or a dedicated processing device (for example, a graphics processing unit (GPU), an application-specific integrated circuit (ASIC), a field-programmable gate array (FPGA), or a programmable logic device). The processor need not be configured by a single processing device but instead may be configured by cooperation of a plurality of processing devices that are present at physically separate positions. In addition, each of the above-described units may be implemented by cooperation of hardware such as a processor, and software.

A communication interface 40 is configured by, for example, a network adapter. The communication interface 40 exhibits a function of communicating with another device (particularly, the camera 12). In particular, the communication interface 40 receives the captured images (captured image for detection 24A and captured image for display 24B) from the camera 12. As described above, in the present embodiment, the communication interface 40 functions as a captured image acquisition unit.

An input interface 42 is configured by, for example, a button, a trackball, or a touch panel. The input interface 42 is used to input a command from an operator, such as a doctor who uses the ultrasound diagnostic apparatus 16, to the ultrasound diagnostic apparatus 16.

A memory 44 includes a hard disk drive (HDD), a solid-state drive (SSD), an embedded MultiMediaCard (eMMC), a read-only memory (ROM), a random-access memory (RAM), or the like. The memory 44 stores a procedure support program for operating each unit of the ultrasound diagnostic apparatus 16. In addition, the procedure support program can also be stored in a non-transitory computer-readable storage medium such as a Universal Serial Bus (USB) memory or a CD-ROM. The ultrasound diagnostic apparatus 16 can read the procedure support program from such a storage medium and execute the procedure support program. Since the ultrasound diagnostic apparatus 16 reads the procedure support program to exhibit the functions described later, the ultrasound diagnostic apparatus 16 can be said to be a computer program product.

The controller 46 includes at least one of a general-purpose processor (such as a CPU) or a dedicated processor (such as a GPU, an ASIC, an FPGA, or a programmable logic device). The controller 46 need not be configured by a single processing device but instead may be configured by cooperation of a plurality of processing devices that are present at physically separate positions. The controller 46 controls each unit of the ultrasound diagnostic apparatus 16. In addition, as shown in FIG. 4, the controller 46 exhibits functions as a probe position and orientation information acquisition unit 48, a volume data acquisition unit 50, a procedure target region specifying unit 52, a reconstruction processing unit 54, and a distance measurement unit 56 in accordance with the procedure support program stored in the memory 44.

The probe position and orientation information acquisition unit 48 acquires position and orientation information indicating the position and the orientation of the ultrasound probe 14. In particular, the probe position and orientation information acquisition unit 48 acquires the position and orientation information indicating the position and orientation of the ultrasound probe 14 in a case in which each frame data for forming ultrasound volume data, which will be described later, is acquired.

In the present embodiment, the probe position and orientation information acquisition unit 48 acquires the position and orientation information by analyzing the captured image for detection 24A acquired by the camera 12 to detect the position and the orientation of the ultrasound probe 14. As described above, the captured image for detection 24A includes the image of the probe detection marker 20 for detecting the position and the orientation of the ultrasound probe 14 (see FIG. 2). The probe position and orientation information acquisition unit 48 acquires the position and orientation information by analyzing the image of the probe detection marker 20 in the captured image for detection 24A. The position of the ultrasound probe 14 may be expressed by, for example, three-dimensional coordinates in a camera coordinate system. The orientation of the ultrasound probe 14 may be expressed by, for example, a rotation angle with respect to each of three predetermined orthogonal axes in the camera coordinate system. Since a known method can be used as a method of detecting the position and the orientation of the ultrasound probe 14 in the camera coordinate system from the image of the probe detection marker 20 included in the captured image for detection 24A, the detailed descriptions thereof will be omitted here.

As described above, the captured image for detection 24A also includes the image of the body surface detection marker 22 for detecting the position and the orientation of the body surface of the subject E (see FIG. 2). The probe position and orientation information acquisition unit 48 may detect the position and the orientation of the body surface of the subject E by analyzing the image of the body surface detection marker 22 in the captured image for detection 24A. In addition, the probe position and orientation information acquisition unit 48 may detect a position and an orientation of the ultrasound probe 14 relative to the position and the orientation of the body surface of the subject E. As a result, the position and the orientation of the ultrasound probe 14 relative to the subject E, which compensate for any variation in the position or the orientation of the subject E, can be obtained.

The probe position and orientation information acquisition unit 48 may detect the position and the orientation of the ultrasound probe 14 by using a method other than analyzing the captured image for detection 24A. For example, a position and orientation sensor such as a magnetic sensor or an acceleration sensor may be provided in the ultrasound probe 14, and the position and the orientation of the ultrasound probe 14 may be detected based on detection values of the position and orientation sensor.

A position and an orientation of the scanning plane scanned with the ultrasound beam are determined by the position and the orientation of the ultrasound probe 14. Therefore, it can be said that the position and orientation information acquired by the probe position and orientation information acquisition unit 48 indicates the position and the orientation of the scanning plane corresponding to each frame data for forming the ultrasound volume data. In addition, each position (coordinates) on the frame data can be specified based on the position and the orientation of the ultrasound probe 14 in a case in which the frame data is acquired. Therefore, it can be said that each frame data is attached with coordinate information indicating each position (coordinates) of the frame data with reference to the position and the orientation of the ultrasound probe 14 in a case in which the frame data is acquired.

The probe position and orientation information acquisition unit 48 stores the frame data acquired by the transmission and reception unit 30 in the memory 44 in association with the position and orientation information indicating the position and the orientation of the ultrasound probe 14 in a case in which the frame data is acquired. As a result, a frame data sequence including a plurality of frame data, each of which is associated with the position and orientation information, is stored in the memory 44.

The volume data acquisition unit 50 acquires the ultrasound volume data, corresponding to the ultrasound irradiation region in the subject E including the procedure target tissue, which is formed based on the reception signals obtained by transmitting and receiving the ultrasound in the ultrasound irradiation region using the ultrasound probe 14 in contact with the body surface portion of the subject E.

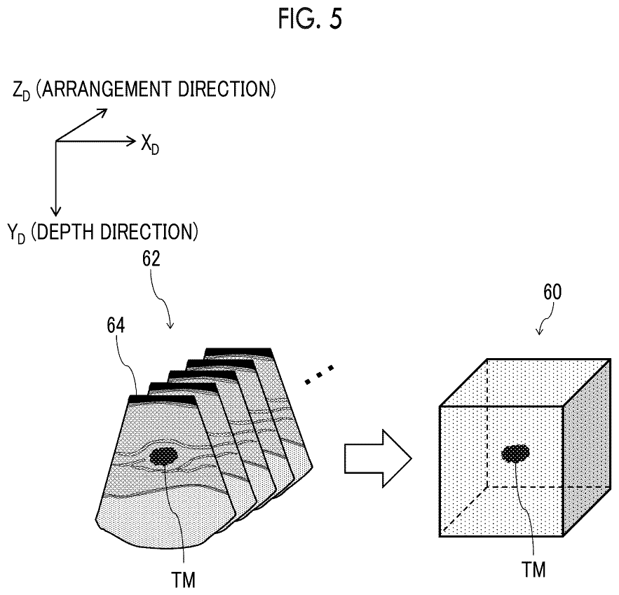

FIG. 5 is a conceptual diagram showing a concept of processing of forming ultrasound volume data 60. In the present embodiment, the volume data acquisition unit 50 forms the ultrasound volume data 60 based on a frame data sequence 62 stored in the memory 44. The frame data sequence 62 is composed of a plurality of frame data 64. Each frame data 64 is data corresponding to the scanning plane of the ultrasound probe 14. Specifically, each frame data 64 is obtained by transmitting and receiving the ultrasound to and from the subject E (particularly, the procedure target tissue) while the ultrasound probe 14 in contact with the subject E is moved by the operator in a sweep direction perpendicular to the scanning plane. In particular, in the present embodiment, since the procedure target tissue is the tumor TM in the breast, the ultrasound probe 14 is brought into contact with the breast of the subject E such that the depth direction of the scanning plane of the ultrasound probe 14 is substantially perpendicular to a coronal plane of the subject E (in other words, substantially parallel to a front-rear direction of the subject E). At least a part of the frame data 64 included in each frame data sequence 62 includes data (a signal indicating the signal intensity of the reflected wave from the tumor TM) related to the tumor TM.

FIG. 5 shows an X_{D} axis direction, a Y_{D} axis direction, and a Z_{D} axis direction, which are orthogonal to each other in a data space in which the ultrasound volume data 60 is defined. In the example of FIG. 5, the volume data acquisition unit 50 first makes the direction of each frame data 64 parallel to an X_{D}Y_{D} plane. In particular, the depth direction of the frame data 64 is made parallel to the Y_{D} axis. Then, the frame data 64 is arranged and combined in an arrangement direction (Z_{D} axis direction in the example of FIG. 5) which is perpendicular to the X_{D}Y_{D} plane to form the ultrasound volume data 60. Since a known method can be used as a method of forming the ultrasound volume data 60 from the plurality of frame data 64, the detailed description thereof will be omitted here.

The frame data 64 for forming the ultrasound volume data 60 may be the plurality of reception beam signals (or a plurality of reception beam signals that have been processed by the signal processing unit 32), corresponding to one scanning plane, which are formed by the transmission and reception unit 30, or may be one ultrasound tomographic image formed by the image formation unit 34 based on the plurality of reception beam signals.

As described above, each frame data 64 is associated with the position and orientation information indicating the position and the orientation of the ultrasound probe 14 in a case in which the frame data 64 is acquired, and has coordinate information indicating each position of each frame data 64 based on the position and the orientation of the ultrasound probe 14. Therefore, the ultrasound volume data 60 including the plurality of frame data 64 also has coordinate information indicating each position of the ultrasound volume data 60 with reference to the position and the orientation of the ultrasound probe 14 in a case in which each frame data 64 is acquired. In the present embodiment, each position of the ultrasound volume data 60 is also expressed by the three-dimensional coordinates in the camera coordinate system. In addition, since the data related to the tumor TM, which is the procedure target tissue, is included in at least a part of the frame data 64 included in each frame data sequence 62, the ultrasound volume data 60 also includes the data related to the tumor TM.

In the present embodiment, the ultrasound volume data 60 is configured of the plurality of frame data 64, but a 2D array probe in which the transducer elements are arranged in two dimensions may be used as the ultrasound probe 14, and the volume data acquisition unit 50 may directly form the ultrasound volume data 60 based on a three-dimensional reception signal from the ultrasound probe 14. Even in this case, the ultrasound volume data 60 includes the coordinate information indicating each position of the ultrasound volume data 60 based on the position and the orientation of the ultrasound probe 14 in a case in which the three-dimensional reception signal is acquired.

The procedure target region specifying unit 52 specifies a procedure target region, which is occupied by the procedure target tissue (tumor TM in the present embodiment) in the ultrasound volume data 60. As a method of specifying the procedure target region, the procedure target region specifying unit 52 can use a learning model that has been trained to identify each tissue (for example, an organ or a tumor) from the ultrasound volume data 60, with the ultrasound volume data 60 as an input. Alternatively, the operator may input an instruction to specify the procedure target region in the ultrasound volume data 60 by displaying the ultrasound volume data 60 on the display 38 with the display controller 36 or the like, and the procedure target region specifying unit 52 may specify the procedure target region in accordance with the instruction.

The reconstruction processing unit 54 forms the reconstructed ultrasound image by determining the cross section in the ultrasound volume data 60 and cutting out the ultrasound volume data 60 in the determined cross section to reconstruct the ultrasound volume data 60.

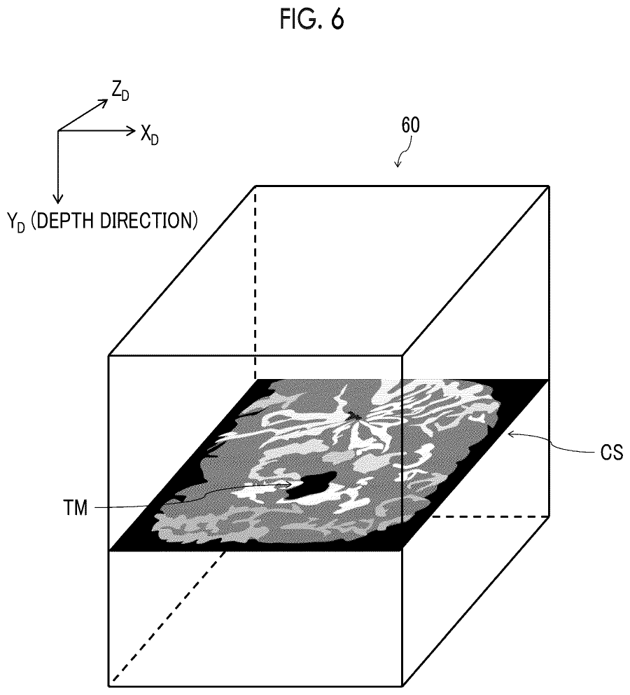

FIG. 6 is a diagram showing a cutout cross section CS in the ultrasound volume data 60. In the present embodiment, the reconstruction processing unit 54 cuts out the ultrasound volume data 60 in the cutout cross section CS perpendicular to the depth direction (Y_{D} axis direction) of the ultrasound volume data 60 and including the procedure target region (in the present embodiment, the region occupied by the tumor TM), to reconstruct the ultrasound volume data 60.

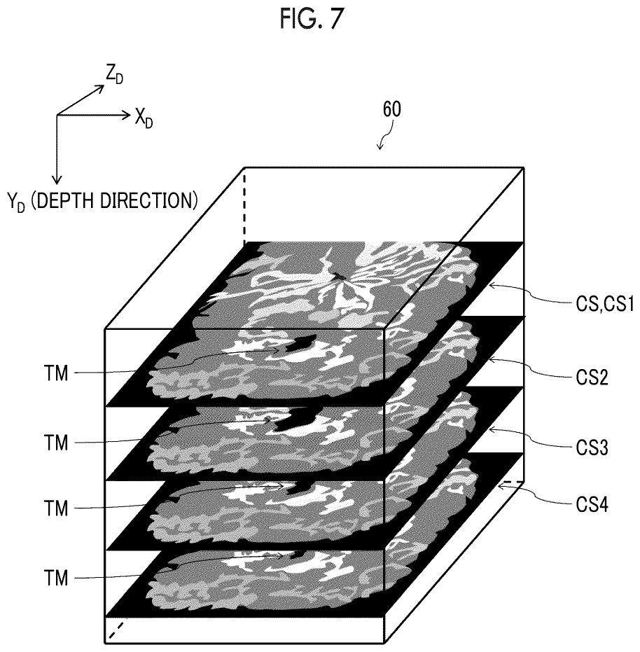

FIG. 7 is a diagram showing a plurality of cutout cross sections CS. As shown in FIG. 7, the plurality of cutout cross sections CS perpendicular to the depth direction of the ultrasound volume data 60 and including the tumor TM can be defined (for example, CS1 to CS4 in FIG. 7). Here, the reconstruction processing unit 54 may determine the position of the cutout cross section CS in the depth direction as a position at which an area of the tumor TM in the cutout cross section CS is the largest. As described above, since the procedure target region specifying unit 52 specifies the region occupied by the tumor TM in the ultrasound volume data 60, the reconstruction processing unit 54 can calculate the area of the tumor TM in the cutout cross section CS. By determining the position of the cutout cross section CS in the depth direction as the position at which the area of the tumor TM is the largest in the cutout cross section CS, it is possible to form the reconstructed ultrasound image in which the tumor TM is represented in a larger size.

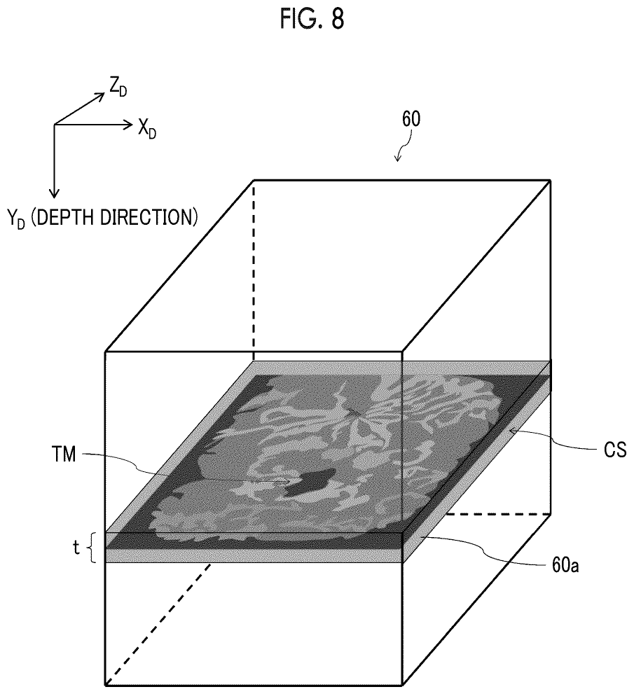

FIG. 8 is a diagram showing a data portion used to form the reconstructed ultrasound image. The reconstruction processing unit 54 may form the reconstructed ultrasound image based on partial volume data 60a, which is a part of the ultrasound volume data 60, includes the cutout cross section CS (preferably, the cutout cross section CS in which the area of the tumor TM is the largest), and has a thickness t in the depth direction of the ultrasound volume data 60. The reconstructed ultrasound image having a stereoscopic effect can be formed by forming the reconstructed ultrasound image based on the partial volume data 60a having the thickness t in the depth direction. An image quality may be degraded due to blurring of the reconstructed ultrasound image in a case in which the thickness t is too large, and the stereoscopic effect is reduced in a case in which the thickness t is too small, and thus the reconstruction processing unit 54 may appropriately set the thickness t. For example, the thickness t may be set based on a size of the tumor TM in the depth direction.

FIG. 9 is a diagram showing an example of a reconstructed ultrasound image 70. The reconstructed ultrasound image 70 is the ultrasound tomographic image including the tumor TM which is the procedure target tissue. As described above, the cutout cross section CS is a plane perpendicular to the depth direction of the ultrasound volume data 60, and thus the reconstructed ultrasound image 70 is an image showing a cross section of the subject E viewed from the depth direction. In the present embodiment, as described above, since the depth direction of the ultrasound volume data 60 is substantially perpendicular to the coronal plane of the subject E, and the cutout cross section CS is also substantially perpendicular to the coronal plane of the subject E, the reconstructed ultrasound image 70 is an image showing the cross section viewed from the depth direction, that is, the cross section parallel to the coronal plane of the subject E.

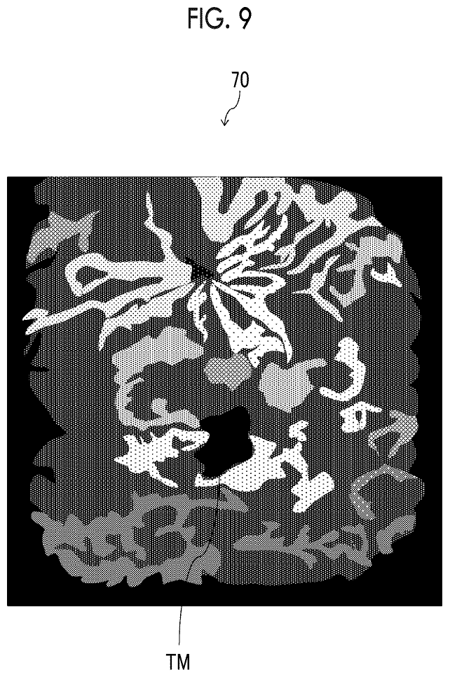

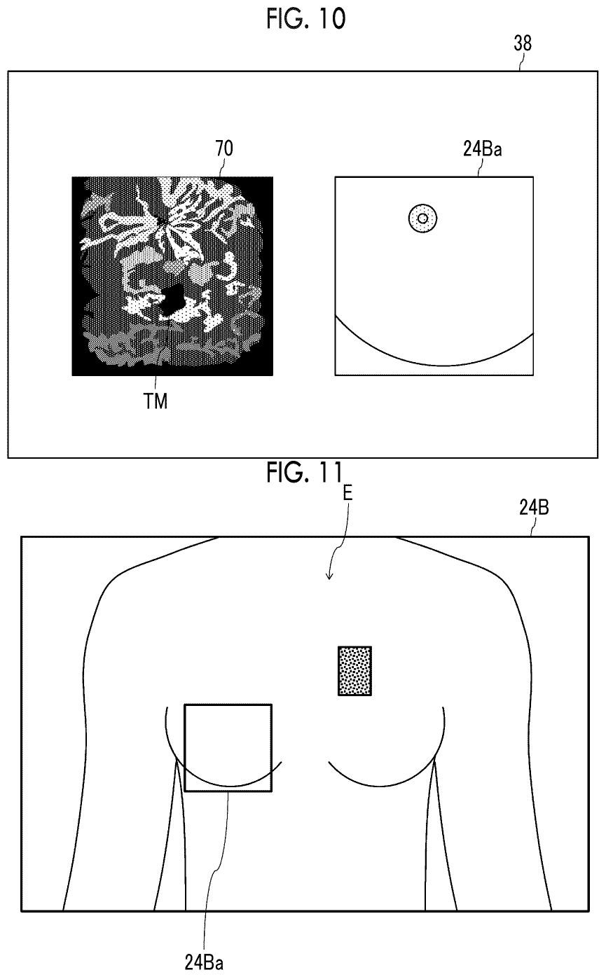

FIG. 10 is a diagram showing a display example of the captured image for display 24B and the reconstructed ultrasound image 70. The display controller 36 displays the captured image for display 24B and the reconstructed ultrasound image 70 on the display 38. As described above, the captured image for display 24B is obtained by imaging the body surface of the subject E (in other words, an optical axis of the lens of the camera 12 is substantially parallel to the depth direction of the subject E), and the reconstructed ultrasound image 70 is an image representing the cross section viewed from the depth direction, and thus the captured image for display 24B and the reconstructed ultrasound image 70 are images viewed from the same visual line direction. In the present embodiment, since the captured image for display 24B is an image obtained by imaging the subject E from the front side, and the reconstructed ultrasound image 70 is an image showing the cross section parallel to the coronal plane of the subject E, both the captured image for display 24B and the reconstructed ultrasound image 70 are images obtained by viewing the subject E from the front side.

Since the captured image for display 24B is obtained by imaging the body surface of the subject E, the position of the tumor TM is not naturally shown in the captured image for display 24B. On the other hand, the position of the tumor TM is shown in the reconstructed ultrasound image 70. Therefore, the operator can specify the position corresponding to the tumor TM on the body surface of the subject E by comparing the reconstructed ultrasound image 70 and the captured image for display 24B, which are the images viewed in the same visual line direction, with each other. That is, the operator can easily identify the incision position on the body surface of the subject E for performing the procedure on the tumor TM in the subject E.

In addition, in a case in which the tumor TM is cancerous, a peripheral tissue of the tumor TM may be pulled to the tumor side and deformed unnaturally. Such deformation is also called “tethering”. With the reconstructed ultrasound image 70, the operator can easily check such a “tethered” state.

The display controller 36 may display the entire captured image for display 24B and the reconstructed ultrasound image 70 on the display 38, but the display controller 36 may preferably display, as shown in FIG. 10, a partial capture image 24Ba, which is a portion of the captured image for display 24B corresponding to the reconstructed ultrasound image 70, and the reconstructed ultrasound image 70 on the display 38.

FIG. 11 is a diagram showing the partial capture image 24Ba in the captured image for display 24B. As described above, since the coordinate information representing each position of the ultrasound volume data 60 is the coordinate information in the camera coordinate system, and the coordinate information representing each position of the ultrasound volume data 60 is based on the position and the orientation (that is, the body surface position of the subject E) of the ultrasound probe 14 in a case in which the ultrasound volume data 60 (in the present embodiment, each frame data 64) is acquired, the display controller 36 can specify the partial capture image 24Ba, corresponding to the reconstructed ultrasound image 70, in the captured image for display 24B based on the coordinate information included in the ultrasound volume data 60.

The display controller 36 cuts out the specified partial capture image 24Ba from the captured image for display 24B, and displays the partial capture image 24Ba and the reconstructed ultrasound image 70 on the display 38 as shown in FIG. 10. It is preferable that the partial capture image 24Ba and the reconstructed ultrasound image 70 may be displayed side by side. Since the partial capture image 24Ba is an image representing a portion corresponding to the reconstructed ultrasound image 70 (the reconstructed ultrasound image 70 represents an internal structure of the portion represented by the partial capture image 24Ba), the operator can more easily specify a position corresponding to the tumor TM on the body surface of the subject E.

In order to make a correspondence relationship between the partial capture image 24Ba and the reconstructed ultrasound image 70 more understandable, the display controller 36 may display the partial capture image 24Ba and the reconstructed ultrasound image 70 on the display 38 with matched display directions and display sizes thereof.

In a case in which the display directions and the display sizes of the partial capture image 24Ba and the reconstructed ultrasound image 70 are made to match each other, it is possible to specify the position of the tumor TM in the partial capture image 24Ba based on the position of the tumor TM in the reconstructed ultrasound image 70. For example, the display controller 36 can specify coordinates of a pixel group corresponding to the tumor TM in the reconstructed ultrasound image 70, and set the pixel group indicated by the same coordinates in the partial capture image 24Ba as the position of the tumor TM.

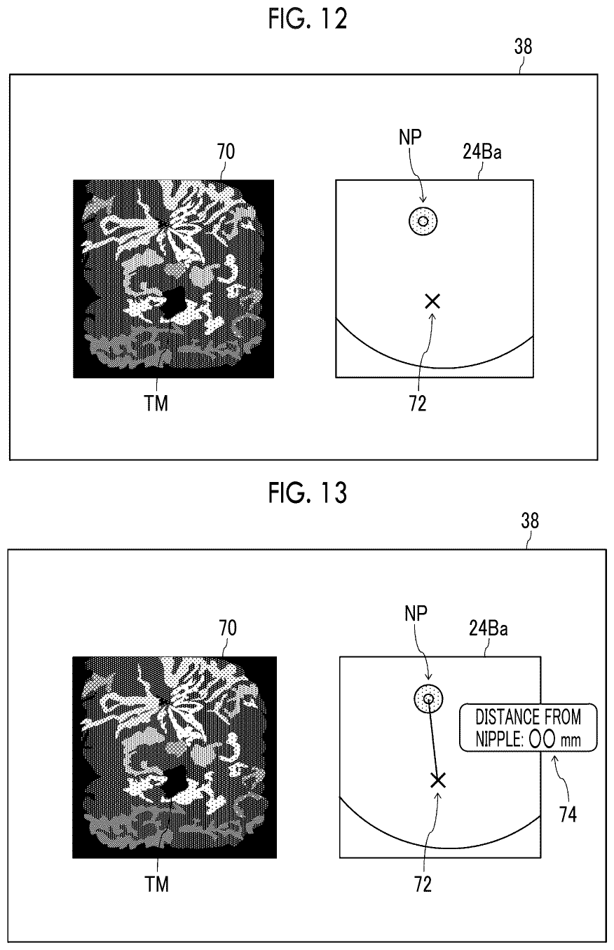

Therefore, as shown in FIG. 12, the display controller 36 may display, on the display 38, a position indicator 72 indicating a position in the partial capture image 24Ba corresponding to the position of the tumor TM in the reconstructed ultrasound image 70. In the example of FIG. 12, an X mark as the position indicator 72 is displayed in a superimposed manner on the position of the tumor TM in the partial capture image 24Ba. As a result, the operator can more easily specify the position corresponding to the tumor TM on the body surface of the subject E.

The operator may determine the incision position on the body surface with reference to a body surface characteristic object on the body surface of the subject E. For example, the incision position is a position of ○○ millimeters from the body surface characteristic object to a foot side. Therefore, in a case in which the image of the body surface characteristic object is included in the partial capture image 24Ba, the distance measurement unit 56 may measure a distance between the body surface characteristic object in the partial capture image 24Ba and the position indicated by the position indicator 72 in the real space (referred to as a “recommended incision position” in the present specification), based on the distance between the image of the body surface characteristic object and the position indicator 72 in the partial capture image 24Ba.

Processing performed by the distance measurement unit 56 will be described with reference to FIG. 12. In the present embodiment, it is assumed that the body surface characteristic object is a nipple NP. First, the distance measurement unit 56 detects an image of the nipple NP from the partial capture image 24Ba. The distance measurement unit 56 can detect the image of the nipple NP from the partial capture image 24Ba by using, for example, a known object detection technique (for example, a learning model such as a regional convolutional neural network (R-CNN)). Then, the distance measurement unit 56 calculates a distance between the image of the nipple NP and the position indicator 72 in the partial capture image 24Ba. The distance is calculated from the coordinates of the image of the nipple NP (for example, a representative position (center position or the like) thereof) in the partial capture image 24Ba and the coordinates of the position indicator 72.

The conversion of the distance between the image of the nipple NP and the position indicator 72 in the partial capture image 24Ba into the distance between the nipple NP and the recommended incision position in the real space can also be performed using a known technique. Specifically, the distance between the nipple NP and the recommended incision position in the real space can be calculated based on a focal length of the camera 12, a distance from the camera 12 to the body surface of the subject E, and the distance between the image of the nipple NP and the position indicator 72 in the partial capture image 24Ba.

The display controller 36 displays the distance between the body surface characteristic object and the position indicated by the position indicator in the real space, which is measured by the distance measurement unit 56, on the display 38. FIG. 13 is a diagram showing a display example of the distance between the nipple NP and the recommended incision position. In the example of FIG. 13, the display controller 36 displays a message window 74 indicating the distance from the nipple in the vicinity of the partial capture image 24Ba.

The ultrasound diagnostic apparatus 16 can perform Doppler measurement for measuring tissue or a velocity of a blood flow of the subject E. The Doppler measurement measures the tissue or the velocity of the blood flow of the subject based on a difference between a frequency of transmission waves from the ultrasound probe 14 and a frequency of reflected waves from the tissue or blood flow of the subject. In the present embodiment, attention is paid to a fact that the blood flow can be measured by the Doppler measurement.

The transmission and reception unit 30 can transmit the transmission waves for Doppler measurement, together with the transmission waves for acquiring the frame data 64 for forming the ultrasound volume data 60. For example, the transmission waves for acquiring the frame data 64 and the transmission waves for the Doppler measurement can be alternately transmitted. As a result, a Doppler signal representing the velocity of the blood flow at each position of the scanning plane can be obtained together with the frame data 64. Therefore, the volume data acquisition unit 50 can acquire the ultrasound volume data 60 including the Doppler signal indicating the velocity of the blood flow of the subject E at each position.

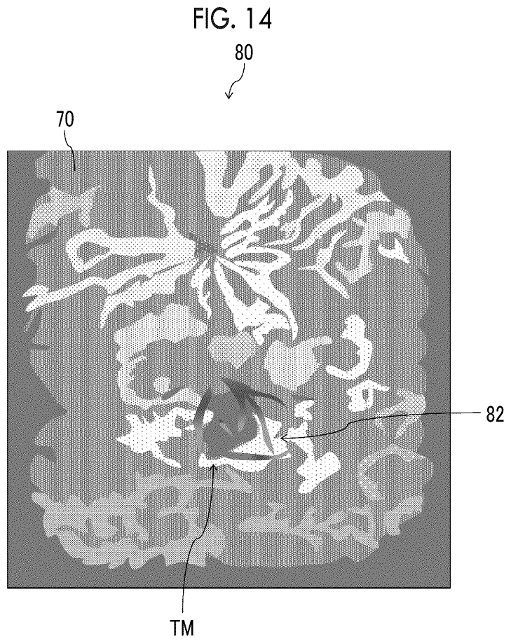

FIG. 14 is a diagram showing an example of a reconstructed Doppler image 80. The reconstruction processing unit 54 cuts out the ultrasound volume data 60 including the Doppler signal in the cutout cross section CS (see FIG. 6) to form the reconstructed ultrasound image 70, and forms a blood flow image 82 representing the blood flow of the subject E in the cutout cross section CS based on the Doppler signal included in the ultrasound volume data 60. The blood flow image 82 may be, for example, a color Doppler image in which the direction and the velocity of the blood flow of the subject E are represented by a hue. Then, the reconstruction processing unit 54 forms an image (which will be referred to as a “reconstructed Doppler image 80” in the present specification) in which the blood flow image 82 is superimposed on the reconstructed ultrasound image 70.

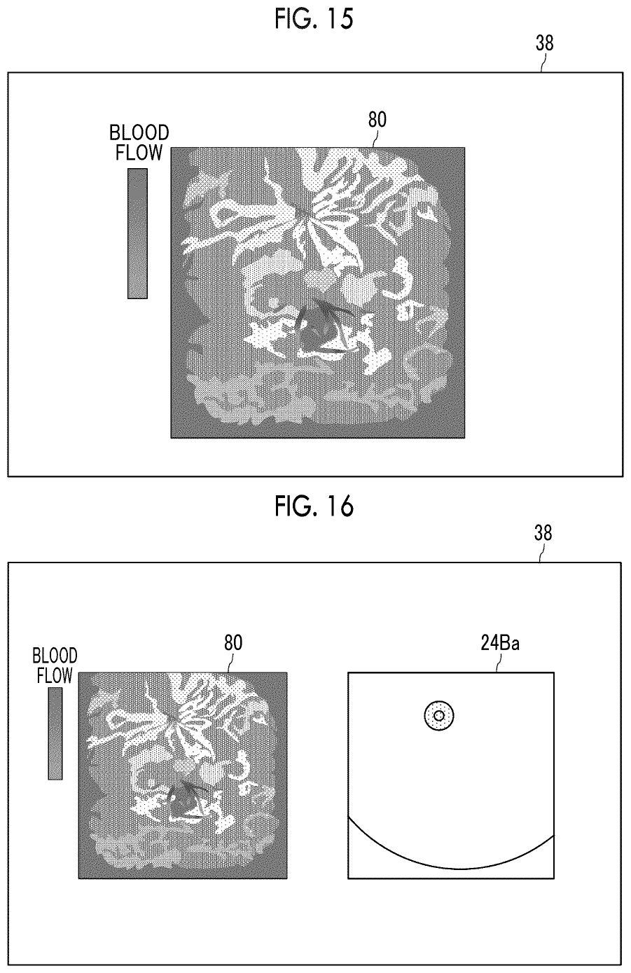

FIG. 15 is a diagram showing a display example of the reconstructed Doppler image 80. The display controller 36 displays the reconstructed Doppler image 80 on the display 38. It is known that a blood flow rate is increased in the tumor TM, particularly in a periphery of a cancerous tumor. Therefore, the operator can check the reconstructed Doppler image 80 and suspect that there is cancer in a case in which there is a portion in which the blood flow rate (≈blood flow velocity) is large. In particular, since cancer metastasizes, in a case in which the reconstructed Doppler image 80 representing the blood flow rate in the vicinity of a location in which cancer is present is displayed after performing a cancer resection procedure, it is possible to support the operator in checking whether or not the cancer that was supposed to have been resected remains or whether or not the cancer has metastasized to the periphery.

As shown in FIG. 16, the display controller 36 may display the reconstructed Doppler image 80 and the captured image for display 24B (particularly, the partial capture image 24Ba) side by side. In this case as well, the partial capture image 24Ba and the reconstructed Doppler image 80 may be displayed on the display 38 with matched display directions and display sizes thereof.

Although the procedure support apparatus according to the present disclosure has been described above, the procedure support apparatus according to the present disclosure is not limited to the above-described embodiment, and various changes can be made without departing from the gist of the present disclosure.

For example, in the above-described embodiment, the procedure support apparatus is the ultrasound diagnostic apparatus 16, and each of the functions of the display controller 36, the volume data acquisition unit 50, the procedure target region specifying unit 52, the reconstruction processing unit 54, and the distance measurement unit 56 is included in the ultrasound diagnostic apparatus 16, but each of these functions need not necessarily be performed by the ultrasound diagnostic apparatus 16. For example, these functions may be exhibited by a server computer or the like that is connected to the ultrasound diagnostic apparatus 16 in a communicable manner. In that case, the processor of the server computer or the like as the procedure support apparatus may acquire the ultrasound volume data 60 from the ultrasound diagnostic apparatus or the like (exhibit the function as the volume data acquisition unit 50), specify the procedure target region in the ultrasound volume data 60 (exhibit the function as the procedure target region specifying unit 52), form the reconstructed ultrasound image 70 from the ultrasound volume data 60 (exhibit the function as the reconstruction processing unit 54), and display the reconstructed ultrasound image 70 on the display that can be viewed by the operator (exhibit the function as the display controller 36). Further, all of the above-described functions may not be exhibited by one device, and the above-described functions may be exhibited by cooperation of a plurality of devices.

What is claimed is:

1. A procedure support apparatus comprising:
a captured image acquisition unit that acquires a captured image for display, which is obtained by imaging a body surface portion in a vicinity of a procedure target tissue in a subject;
a volume data acquisition unit that acquires ultrasound volume data, corresponding to an ultrasound irradiation region including the procedure target tissue in the subject, which is formed based on reception signals obtained by transmitting and receiving ultrasound in the ultrasound irradiation region using an ultrasound probe in contact with the body surface portion;
a procedure target region specifying unit that specifies a procedure target region that is occupied by the procedure target tissue in the ultrasound volume data;
a reconstruction processing unit that forms a reconstructed ultrasound image by cutting out the ultrasound volume data in a cutout cross section perpendicular to a depth direction of the ultrasound volume data and including the procedure target region to reconstruct the ultrasound volume data; and
a display controller that displays the captured image for display and the reconstructed ultrasound image on a display unit.

2. The procedure support apparatus according to claim 1, further comprising:
a probe position and orientation information acquisition unit that acquires position and orientation information indicating a position and an orientation of the ultrasound probe in a case in which the ultrasound volume data is acquired,
wherein the captured image acquisition unit acquires a captured image for detection, which is obtained by imaging a probe detection marker attached to the ultrasound probe in contact with the body surface portion,
the volume data acquisition unit acquires the ultrasound volume data including coordinate information indicating each position of the ultrasound volume data with reference to the position and orientation information in a camera coordinate system of a camera that has captured the captured image for display, and
the display controller displays a partial capture image that is a portion of the captured image for display corresponding to the reconstructed ultrasound image on the display unit based on the coordinate information included in the ultrasound volume data.

3. The procedure support apparatus according to claim 2,
wherein the display controller displays the partial capture image and the reconstructed ultrasound image with matched display directions and display sizes thereof on the display unit.

4. The procedure support apparatus according to claim 3,
wherein the display controller displays a position indicator indicating a position in the partial capture image corresponding to a position of the procedure target region in the reconstructed ultrasound image on the display unit.

5. The procedure support apparatus according to claim 4,
wherein the partial capture image includes an image of a body surface characteristic object on a body surface of the subject,
the procedure support apparatus further comprises a distance measurement unit that measures a distance between the body surface characteristic object and the position indicated by the position indicator in a real space, based on a distance between the image of the body surface characteristic object and the position indicator in the partial capture image, and
the display controller displays the distance measured by the distance measurement unit on the display unit.

6. The procedure support apparatus according to claim 1,
wherein the reconstruction processing unit determines a position of the cutout cross section in the depth direction as a position at which an area of the procedure target region in the cutout cross section is largest.

7. The procedure support apparatus according to claim 1,
wherein the reconstruction processing unit forms the reconstructed ultrasound image based on partial volume data that is a part of the ultrasound volume data, including the cutout cross section and having a thickness in the depth direction of the ultrasound volume data.

8. The procedure support apparatus according to claim 1,
wherein the volume data acquisition unit acquires the ultrasound volume data including a Doppler signal indicating a velocity of a blood flow of the subject at each position,
the reconstruction processing unit forms a blood flow image representing the blood flow of the subject in the cutout cross section based on the Doppler signal, and forms a reconstructed Doppler image in which the blood flow image is superimposed on the reconstructed ultrasound image, and
the display controller displays the reconstructed Doppler image on the display unit.

9. A procedure support apparatus comprising:
a volume data acquisition unit that acquires ultrasound volume data, corresponding to an ultrasound irradiation region including a procedure target tissue in a subject, which is formed based on reception signals obtained by transmitting and receiving ultrasound in the ultrasound irradiation region using an ultrasound probe in contact with a body surface portion in a vicinity of the procedure target tissue in the subject, the ultrasound volume data including a Doppler signal indicating a velocity of a blood flow of the subject at each position;
a procedure target region specifying unit that specifies a procedure target region corresponding to the procedure target tissue in the ultrasound volume data;
a reconstruction processing unit that forms a reconstructed ultrasound image by cutting out the ultrasound volume data including the Doppler signal in a cutout cross section perpendicular to a depth direction of the ultrasound volume data and including the procedure target region to reconstruct the ultrasound volume data, that forms a blood flow image representing the blood flow of the subject in the cutout cross section based on the Doppler signal, and that forms a reconstructed Doppler image in which the blood flow image is superimposed on the reconstructed ultrasound image; and
a display controller that displays the reconstructed Doppler image on a display unit.

10. A non-transitory computer-readable storage medium storing a procedure support program causing a computer to function as:
a captured image acquisition unit that acquires a captured image for display, which is obtained by imaging a body surface portion in a vicinity of a procedure target tissue in a subject;
a volume data acquisition unit that acquires ultrasound volume data, corresponding to an ultrasound irradiation region including the procedure target tissue in the subject, which is formed based on reception signals obtained by transmitting and receiving ultrasound in the ultrasound irradiation region using an ultrasound probe in contact with the body surface portion;
a procedure target region specifying unit that specifies a procedure target region that is occupied by the procedure target tissue in the ultrasound volume data;
a reconstruction processing unit that forms a reconstructed ultrasound image by cutting out the ultrasound volume data in a cutout cross section perpendicular to a depth direction of the ultrasound volume data and including the procedure target region to reconstruct the ultrasound volume data; and
a display controller that displays the captured image for display and the reconstructed ultrasound image on a display unit.
